(19) United States
(12) Patent Application Publication
(10) Pub. No.: US 2005/0095595 A1
(43) Pub. Date: May 5, 2005

(54) Methods and systems for improving of polymer analysis

(76) Inventors: Richard J. Pittaro, San Carlos, CA (US); Douglas A. Amorese, Los Altos, CA (US); Carol T. Schembri, San Mateo, CA (US); Leslie A. Leonard, Portola Valley, CA (US); Nicholas M. Sampas, San Jose, CA (US); Michael P. Caren, Palo Alto, CA (US)

Correspondence Address: AGILENT TECHNOLOGIES, INC.;Legal Department, DL 429, Intellectual Property Administration, P.O. Box 7599, Loveland, CO, 80537-0599, US

(21) Appl. No.: 10/696,177

(22) Filed: Oct. 29, 2003

(51) Int. Cl. C12Q 1/68

(52) U.S. Cl. 435/6

(57) ABSTRACT

Systems and methods for optimizing polymer analysis are provided. One such system includes a polymer analysis system having a polymer control system. The polymer analysis system is operative to: apply a set of conditions to a sample being analyzed by the polymer array, the set of conditions correspond to at least one characteristic of the polymer array; analyze the at least one characteristic using the polymer control system; and generate polymer array data.

### BACKGROUND

Nearly every area of the biomedical sciences uses a system to assay chemical and biochemical reactions to determine the presence and/or quantity of particular analytes. Polymer arrays are used in basic science research laboratories, clinical diagnostics, pharmaceutical research and drug discovery, and DNA and protein analysis. In all of these applications, the presence and/or quantity of a specific analyte or group of analytes, can be determined using polymer array systems.

For analysis in the fields of pharmacology, genetics, chemistry, biochemistry, biotechnology, molecular biology and numerous others, it is often useful to detect the presence of one or more molecular structures and measure interactions between molecular structures. The molecular structures of interest typically include, but are not limited to, cells, antibodies, antigens, metabolites, proteins, drugs, small molecules, enzymes, nucleic acids, and other ligands and analytes. In medicine, for example, it is very useful to determine the existence of cellular constituents such as receptors or cytokines, or antibodies and antigens, which serve as markers for various disease processes, and may exist naturally in physiological fluids or may have been introduced into the system. In genetic analyses, fragment DNA and RNA sequence analysis is very useful in diagnostics, genetic testing and research, agriculture, and pharmaceutical development.

Polymer arrays are tools used by drug researchers and geneticists, which provide information about polynucleotides (e.g., nucleic acid sequences) and polypeptides (e.g., amino acid sequences) in a sample. Polymer arrays include polymer probes attached to a surface to which a sample is added, where the sample may contain polymers corresponding to one or more of the polymer probes.

Polymer arrays can be of the following types: a polynucleotide array and a polypeptide array. For clarity, the following discussion focuses on polynucleotide arrays.

Polynucleotide arrays typically include between a few hundred to over a hundred thousand or more polynucleotide probes in the form of DNA strands arranged in a determined pattern on a substrate. In some cases the polynucleotides are identical, and in other cases the polynucleotides are different. Polynucleotide arrays can be used to determine whether target polynucleotide sequences interact or hybridize with any of the polynucleotide probes on the polynucleotide array. After exposing the polynucleotide array to target polynucleotide sequences under general test conditions, scanning devices can examine each location in the polynucleotide array and determine whether a target polynucleotide has hybridized with the probe polynucleotide at that location. Polynucleotide arrays can be used to determine if genes are “turned on” or up-regulated and if genes are “turned off” or down-regulated. For example, a researcher can compare a normal colon cell with a malignant colon cell and thereby determine which genes are being expressed or not expressed only in the aberrant cell. The regulation of these genes serves as key targets for drug therapy.

Polynucleotide hybridization is a hydrogen-bonding interaction between two polynucleotide strands that obey the Watson-Crick complementary rules. All other base pairs are mismatches that destabilize hybrids. Since a single mismatch decreases the melting temperature of a hybrid by up to 10° C., conditions can be found in which only perfect hybrids can survive. Many hybridization experiments can be simultaneously carried out on a single solid support on which multiple polynucleotide probes have been immobilized by either covalent or non-covalent methods. The polynucleotide probe is hybridized with target polynucleotides that usually bear a radioactive label, fluorescent label, or haptens that can be visualized by chemiluminescent or other detection methods. The resulting hybrid duplexes are separated from the unreacted labeled strands by washing the support. Generally, the hybrid duplexes are recognized by detecting the label bound to the surface of the support.

In performing the hybridization, depending on the reagent (buffer) compositions employed, and the similarity of the probe polynucleotides and target polynucleotides, the temperature employed may vary from about ambient temperature to about 70° C. As described above, temperature is used as a process variable in altering the hybridization stringency. Typically, polynucleotide hybridizations are carried out in a closed container in a constant temperature environment for extended periods of time (e.g., 10-18 hours).

Polynucleotide hybridization is widely used to determine the presence of a polynucleotide sequence that is complimentary to the polynucleotide probe and/or quantify such presence. In many cases, this provides a simple, fast, and inexpensive alternative to conventional sequencing methods. Polynucleotide hybridization does not require polynucleotide cloning and purification, carrying out base-specific reactions, or tedious electrophoretic separations. Polynucleotide hybridization of polynucleotide probes has been successfully used for various purposes, such as analysis of genetic polymorphisms, gene expression, diagnosis of diseases, cancer diagnostics, detection of viral and microbial pathogens, screening of clones, genome mapping and ordering of fragment libraries.

Polynucleotide arrays can contain a chosen collection of polynucleotides (e.g., probe polynucleotides specific for all known clinically important pathogens or specific for all known clinically important pathogens or specific for all known sequence markers of genetic diseases). Such an array can satisfy the needs of a diagnostic laboratory. Alternatively, a polynucleotide array can contain a substantial subset of polynucleotides of a given length to probe all known genes. Hybridization of a nucleic acid with such a comprehensive array results in a list of all its constituent nucleic acids, which can be used for unambiguous gene identification (e.g., in forensic studies), for determination of unknown gene variants and mutations (including the re-sequencing of related genomes once the sequence of that gene is known), for overlapping clones, and for checking sequences determined by conventional methods. Finally, surveying the nucleic acids by hybridization to a comprehensive array may provide sufficient information to determine the sequence of an unknown nucleic acid.

Currently, polymer array analysis is performed under pre-set conditions that focus on the entire polymer array. However, there is a need in the art to specifically optimize polymer array analysis.

### SUMMARY

Systems and methods for improving polymer analysis are provided. One such method comprises: providing a sample and a polymer array, the polymer array having a plurality of spots; providing a set of conditions that are selected to generate a response from selected spots; applying the set of conditions to the sample; and generating data corresponding to the selected spots.

Another method includes: providing a sample and a polymer array, the polymer array having a plurality of spots; providing a set of hybridization conditions and a set of wash conditions that are selected to generate a response for selected spots; applying the hybridization conditions to the sample; generating hybridization data corresponding to the selected spots; adjusting the hybridization conditions applied to the sample until the hybridization data satisfy a hybridization criteria; applying the wash conditions to the sample; generating washing data corresponding to the selected spots; adjusting the washing conditions applied to the sample until the washing data satisfy a washing criteria; and generating polymer array data.

A system for improving polymer analysis comprises a polymer analysis system having a polymer array and a polymer control system. The polymer control system is operative to: apply a set of conditions to a sample being analyzed using a polymer array, the polymer array having a plurality of spots, the set of conditions are selected to generate a response from selected spots; analyze data corresponding to the selected spots using the polymer control system; and generate polymer array data.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

Reference is now made to the following drawings. Note that the components in the drawings are not necessarily to scale.

FIG. 1 is a flowchart depicting functionality associated with an embodiment of a polynucleotide analysis system.

FIG. 2 is a schematic diagram of a computer or processor-based system that can be used to implement an embodiment of a polynucleotide control system.

FIG. 3 is a flowchart depicting functionality of an embodiment of a polynucleotide control system.

FIG. 4 is a flowchart depicting functionality of another embodiment of a polynucleotide control system.

FIG. 5 is a flowchart depicting functionality of still another embodiment of a polynucleotide control system.

### DETAILED DESCRIPTION

As will be described in greater detail here, systems and methods that improve the performance of polymer array analysis are provided. By way of example, some embodiments provide for polymer analysis systems and methods of use thereof, that measure a response for selected spots on a polymer array under tailored conditions. Focusing on the response from the selected spots can enhance the analysis of a disease or condition in a host from which a sample was taken. In addition, other embodiments provide for polymer analysis systems and methods of use thereof, that dynamically measure and adjust the conditions applied to selected spots on the polymer array, which is done to optimize the conditions for generating an enhanced response from the selected spots.

The polymer array can be a polynucleotide or a polypeptide array. The term “polynucleotide” refers to nucleic acid polymers or portions thereof such as, but not limited to, oligonucleotides (e.g., up to 100 nucleotide bases), nucleotides (e.g., greater than 100 nucleotide bases), and deoxyribonucleotide and ribonucleotide polymers in either single- or double-stranded forms. The term “polypeptide” refers to amino acid polymers or portions thereof such as, but not limited to, proteins. For clarity, reference to polynucleotide arrays is made throughout the remainder of this disclosure. However, the methods and systems of this disclosure can be modified and applied to polypeptide arrays and polypeptide analysis systems.

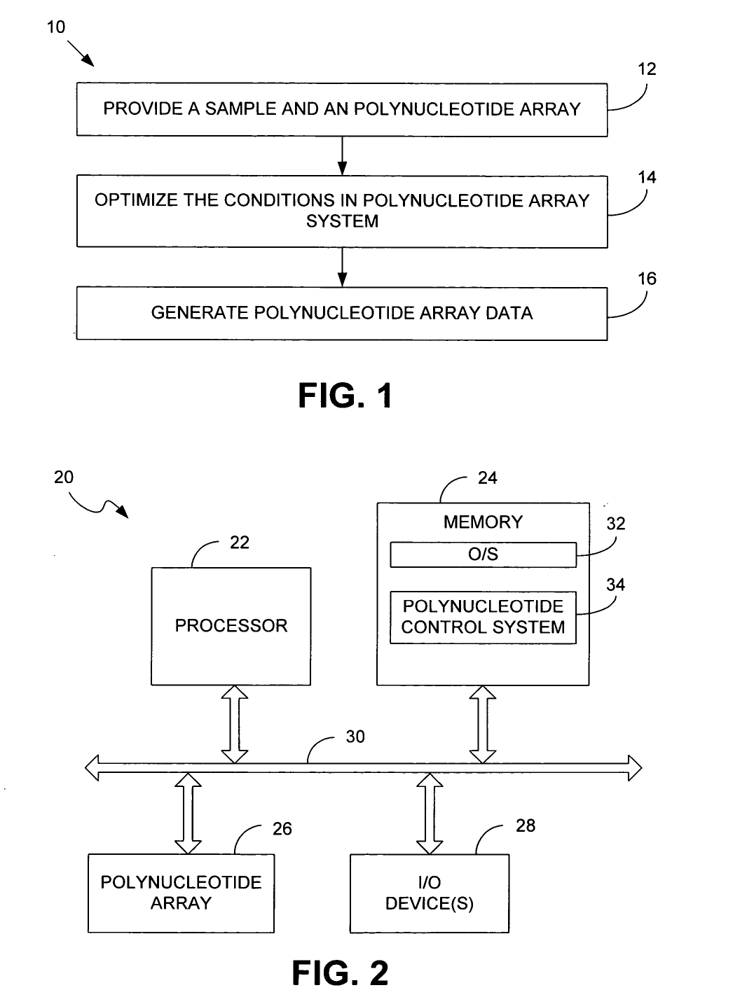

FIG. 1 is a flowchart depicting functionality of an embodiment of a polynucleotide analysis system 10 that can be used for enhancing the analysis of a sample. As shown in FIG. 1, the functionality (or method) may be construed as beginning at block 12, where a sample and a polynucleotide array having a plurality of spots are provided. The sample is introduced to the polynucleotide array to determine whether target polynucleotide sequences of the sample interact (e.g., hybridize) with the polynucleotide probes on selected spots of the polynucleotide array. In block 14, the conditions of the polynucleotide array system are optimized based on a response from the selected spots. In this manner the polymer analysis is targeted towards the hybridization of the target polynucleotide to probe polynucleotides on the selected spots. Therefore, the measured response is specific for only a portion of the polymer array (i.e., the selected spots).

After exposing the polynucleotide array to target polynucleotide sequences under selected conditions to optimize the polynucleotide array analysis, scanning devices can examine each spot in the polynucleotide array and determine the degree to which the target polynucleotide and the probe polynucleotide have hybridized. In particular, the scanning device examines the selected spots of the polymer array. Thereafter, polynucleotide array data corresponding to the sample is generated, as shown in block 16.

As discussed above, the term “hybridization” as used herein involves the annealing of the polynucleotide probes to their corresponding target polynucleotides (the sequence to be detected). The ability of two polynucleotide polymers containing complementary sequences to find each other and anneal through base pairing interaction is a well-recognized phenomenon.

“Probe polynucleotides” include a functional genetic unit such as a portion of a DNA molecule or the entire cDNA molecule. In addition, the probe polynucleotide may, for example, contain specific genes or, be from a chromosomal region suspected of being present at increased or decreased copy number in cells of interests (e.g., tumor cells). Further, the probe polynucleotide may also contain an mRNA, or cDNA derived from such mRNA, suspected of being transcribed at abnormal levels.

Alternatively, a probe polynucleotide may comprise nucleic acids of unknown significance or location. An array (e.g., spots disposed on the polymer array) of such elements could represent locations that sample, either continuously or at discrete points, any desired portion of a genome, including, but not limited to, an entire genome, a single chromosome, or a portion of a chromosome. The probe polynucleotides of the arrays may be arranged on the solid surface at different densities. The number of probe polynucleotides and the complexity of the nucleic acids as well as the nature of the label and the solid support determine the density of sampling.

Similarly, an array of probe polynucleotides (i.e., a polynucleotide array) comprising nucleic acids from anonymous cDNA clones would permit identification of those that might be differentially expressed in some cells of interest, thereby focusing attention on study of these genes.

One of skill in the art should recognize that the polynucleotide arrays may comprise a mixture of probe polynucleotides of different lengths and sequences. Thus, for example, a polynucleotide array may contain more than one copy of a cloned piece of DNA, and each copy may be broken into fragments of different lengths. The length and complexity of the probe polynucleotides is not critical. One of skill in the art can adjust these factors to produce an appropriate signal for a given hybridization procedure, and to provide the required resolution among different genes or genomic locations.

Typically, the functionality described with respect to FIG. 1 is implemented, at least in part, by a polynucleotide analysis system. Embodiments of the polynucleotide analysis system can be implemented in hardware, software and/or combinations thereof. An embodiment of a polynucleotide analysis system that is implemented in software is depicted schematically in FIG. 2, where the polynucleotide analysis system is associated with a computer or processor-based system 20.

Generally, computer 20 includes a processor 22, memory 24, and a polynucleotide array 26, and one or more input and/or output (I/O) devices 28 (or peripherals) that are communicatively coupled via a local interface 30. The software in memory 24 can include one or more separate programs, each of which comprises an ordered listing of executable instructions for implementing logical functions. In the example of FIG. 2, the software in the memory 24 includes an operating system (O/S) 32 and a polynucleotide control system 34.

When the polynucleotide control system 34 is implemented in software, it should be noted that the polynucleotide control system 34 could be stored on any computer-readable medium for use by or in connection with any computer-related system or method. In the context of this document, a computer-readable medium is an electronic, magnetic, optical, or other physical device or means that can contain or store a computer program for use by or in connection with a computer-related system or method. The polynucleotide control system 34 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium. More specific examples (a nonexhaustive list) of the computer-readable medium would include the following: an electrical connection (electronic) having one or more wires, a portable computer diskette (magnetic), a random access memory (RAM) (electronic), a read-only memory (ROM) (electronic), an erasable programmable read-only memory (EPROM, EEPROM, or Flash memory) (electronic), an optical fiber (optical), and a portable compact disc read-only memory (CDROM) (optical). Note that the computer-readable medium could even be paper or another suitable medium upon which the program is printed, as the program can be electronically captured, via for instance optical scanning of the paper or other medium, then compiled, interpreted or otherwise processed in a suitable manner if necessary, and then stored in a computer memory.

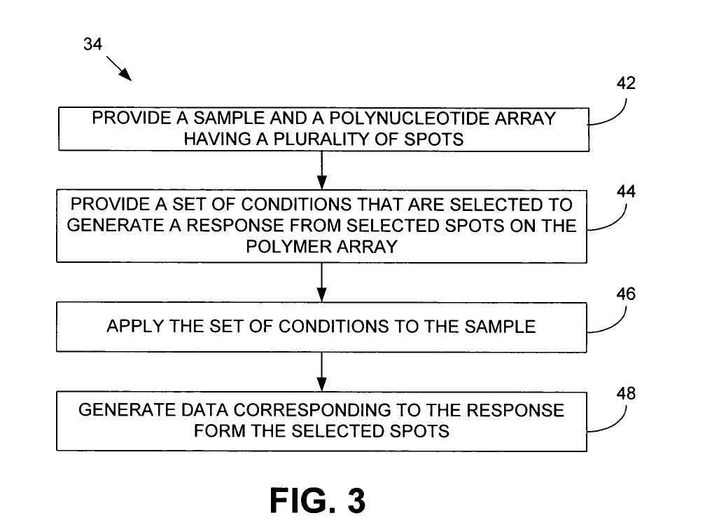

Functionality of the polynucleotide control system 34 of FIG. 2 is depicted in the flowchart of FIG. 3. As shown in FIG. 3, the functionality (or method) may be construed as beginning at block 42, where a sample and a polynucleotide array having a plurality of spots are provided. In block 44, a set of conditions that are selected to generate a response from selected spots on the polymer array is provided, where the selected spots can include target polynucleotides. In block 46, the set of conditions is applied to the sample. In block 48, data corresponding to the response from the selected spots of the polymer array is generated.

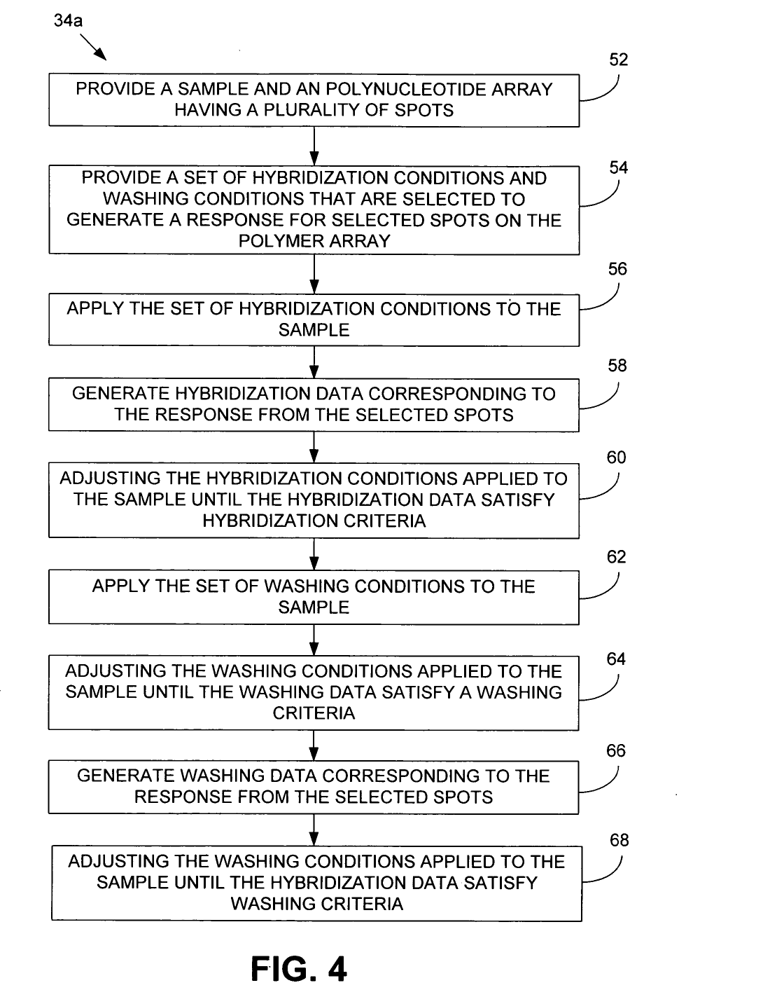

In another embodiment, the functionality of another polynucleotide control system 34a is depicted in the flowchart of FIG. 4. As shown in FIG. 4, the functionality may be construed as beginning at block 52, where a sample and a polynucleotide array having a plurality of spots are provided. In block 54, a set of hybridization conditions and a set of washing conditions that are selected to generate a response from selected spots on the polymer array are provided, where the selected spots can include target polynucleotides. In block 56, the hybridization conditions are applied to the sample and hybridization data corresponding to the response from the selected spots is generated (block 58). Hybridization data includes data that correspond to the response from the selected spots upon application of the hybridization conditions to the polymer array. In block 60, the hybridization conditions applied to the sample are dynamically adjusted until the measured hybridization data satisfy one or more hybridization criteria, which are described in more below.

In block 62, the washing conditions are applied to the sample. In block 64, the washing conditions are applied to the sample and washing data corresponding to the response from the selected spots are generated (block 66). Washing data includes data that correspond to the response from the selected spots upon application of the washing conditions to the polymer array. In block 68, the washing conditions applied to the sample are dynamically adjusted until the washing data satisfy one or more hybridization criteria, which are described in more detail below.

By focusing on the response from the selected spots of the polynucleotide array and optimizing the conditions based on the response from the selected spots, the polynucleotide data corresponding to the selected spots obtained from analysis of the polynucleotide array may be enhanced relative to the data obtained when general array conditions are used (e.g., generalized to obtain polynucleotide data from all of the spots on the polynucleotide array rather than selected spots). In other words, measuring the formation of hybrid duplexes (e.g., the polynucleotide probes and target polynucleotides hybrid duplexes formed on the selected spots) under conditions optimized for formation of the hybrid duplexes enables the speed of the overall polynucleotide array analysis to be increased while also enhancing the specificity and response of the polynucleotide array analysis. This approach is in contrast to generally used techniques that measure the formation of hybrid duplexes on many thousands of polynucleotide probes under general conditions, where the general conditions emphasize the overall performance of the array rather than the performance of selected spots of the polymer array. Therefore, the polynucleotide analysis systems 10 disclosed here can be tailored to focus on target polynucloetides located on selected spots of the polynucleotide array and can dynamically optimize the measurement of the target polynucloetides using specific conditions.

The polynucleotide analysis system 10 can be used to screen for one or more target polynucleotides specific for a disease or condition by optimizing the conditions to which the sample is exposed. The selected spots can include one or more polynucleotides from which data can be obtained. The data can be obtained by measuring responses from polynucleotides such as, but not limited to, one or more trait-specific probe polynucleotides, one or more calibration-specific probe polynucleotides, relationships between two or more trait specific probe polynucleotides, relationships between two or more calibration-specific probe polynucleotides, and relationships between one or more trait-specific probe polynucleotides and one or more calibration-specific probe polynucleotides.

The trait-specific probe polynucleotides include, but are not limited to, polynucleotides that correspond to, relate to, and/or are a known marker for a disease. The calibration-specific probe polynucleotides include, but are not limited to, polynucleotides that correspond to known polynucleotide probes that can be used to evaluate how certain types of polynucleotide probes are responding to the conditions of the polynucleotide array. The relationships (e.g., ratios) between two or more trait-specific probe polynucleotides, two or more calibration-specific probe polynucleotides, and one or more trait-specific probe polynucleotides and one or more calibration-specific probe polynucleotides, can be used to evaluate how certain types of polynucleotides are responding to the conditions applied to the polynucleotide array.

The criteria represent a threshold value or value range to which the measured data (i.e., response of the selected spots) is compared. The measured data values can be altered by adjusting one or more of the conditions applied to the polymer array. Therefore, the conditions can be adjusted to change the response (data) of the selected spots until one or more of the criteria are met. For example, if the measured data exceeds or is less than the criteria values, or is within the range of the criteria values, the selected criteria are satisfied. The criteria can include, but is not limited to, hybridization criteria and washing criteria. In particular, hybridization criteria include threshold values or value ranges that can be compared to measured hybridization data values. The criteria can be compared to measured data values such as, but not limited to, one or more trait-specific probe polynucleotides, one or more calibration-specific probe polynucleotides, relationships between two or more trait specific probe polynucleotides, relationships between two or more calibration-specific probe polynucleotides, and relationships between one or more trait-specific probe polynucleotides and one or more calibration-specific probe polynucleotides.

The conditions can include, but are not limited to, the stringency conditions of the polynucleotide array analysis (e.g., the hybridization conditions and the wash conditions). In particular, these conditions can include, but are not limited to, the temperature of the polynucleotide array, pH of the polynucleotide array, the time period of the polynucleotide array, as well as chemicals that can be added to the polynucleotide array that affects hybridization. Each of these conditions, independently or in combination, can be dynamically adjusted to increase and/or decrease the hybridization of the target polynucleotides to one or more polynucleotide probes.

When used in reference to polynucleotide arrays, it is known in the art that numerous equivalent conditions may be employed to comprise either low, medium, or high stringency conditions; factors such as, but not limited to, the length and nature (DNA, RNA, base composition) of the polynucleotide probe and nature of the target polynucleotide (DNA, RNA, base composition, present in solution or immobilized, etc.), the concentration of the salts, and other components (e.g., the presence or absence of formamide, dextran sulfate, polyethylene glycol) are considered. In addition, the hybridization solution may be varied to generate hybridization conditions of either lower or higher stringency different from, but equivalent to, the above listed conditions. One of skill should recognize that relaxing the stringency of the hybridizing conditions allows sequence mismatches to be tolerated. The degree of mismatch tolerated can be controlled by suitable adjustment of the hybridization conditions. In addition, the wash solution may be varied to generate wash conditions of either lower or higher stringency different from, but equivalent to, the above listed conditions.

It is generally recognized that polynucleotides are denatured by increasing the temperature, decreasing the salt concentration of the buffer containing the nucleic acids, or increasing the pH of the hybridization and/or wash solutions. Under low stringency conditions (e.g., low temperature, and/or high salt concentration, and/or high target polynucleotide concentration) hybrid duplexes (e.g., DNA:DNA, RNA:RNA, or RNA:DNA) form even where the annealed sequences are not perfectly complementary. Thus, specificity of hybridization is reduced at lower stringency. Conversely, at higher stringency (e.g., higher temperature or lower salt concentration) successful hybridization requires fewer mismatches. One of skill in the art should understand that the hybridization conditions and/or the wash conditions could be selected to provide any degree of stringency.

In general, there is a tradeoff between hybridization specificity (stringency) and signal intensity. For example, the hybridization and/or wash can be performed at the highest stringency to produce consistent results and provide signal intensity greater than approximately 10% of the background intensity. In another example, the polynucleotide array may be exposed to hybridization solutions at successively higher stringency solutions. In still another example, the polynucleotide array may be washed with the wash solution at successively higher stringency solutions. Analysis of the respective data sets produces a hybridization and wash stringency for which the hybridization pattern is not appreciably altered and which provides adequate signal for the particular polynucleotide probes of interest.

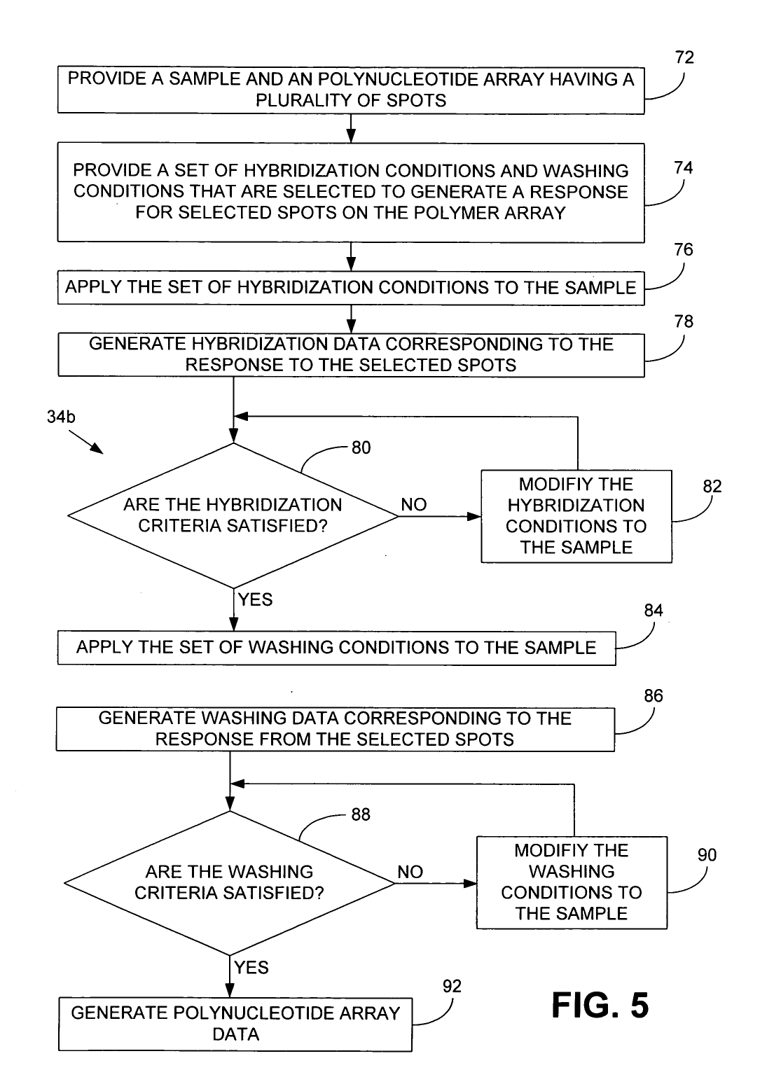

In another embodiment, the functionality of another polynucleotide control system 34b is depicted in the flowchart of FIG. 5. As shown in FIG. 5, the functionality may be construed as beginning at block 72, where a sample and a polynucleotide array having a plurality of spots are provided. In block 74, a set of hybridization conditions and a set of washing conditions that are selected to generate a response from selected spots on the polymer array is provided, where the selected spots can include target polynucleotides. In block 76, the hybridization conditions are applied to the sample and hybridization data corresponding to the response of the selected spots is generated (block 78). In decision block 80, a determination is made as to whether the hybridization criteria are satisfied. If the determination in block 80 is “no,” then the hybridization conditions are modified as shown in block 82. After the hybridization conditions are modified, the flow loops back to block 80. This cycle continues until the hybridization criteria are satisfied.

Once the hybridization criteria are satisfied, the flow continues to block 84, where the washing conditions are applied to the sample. In block 86, washing data corresponding to the response from the selected spots is generated. In block 88, a determination is made as to whether the washing criteria are satisfied. If the determination in block 88 is “no,” then the washing conditions are modified, as shown in block 90. After the hybridization conditions are modified, the process returns to block 88. This cycle continues until the washing criteria are satisfied. Once the washing criteria are satisfied, the process continues to block 92, where polynucleotide array data is generated.

As described above, the response can be dynamically measured and analyzed so that the conditions can be adjusted to satisfy one or more sets of criteria. In other words, a feedback loop can be used to measure the hybridization response of the polynucleotide probes and the target polynucleotides, and if the response does not satisfy certain criteria, then the conditions can be adjusted in a manner to change the hybridization to satisfy the criteria. This feedback loop can be performed until the criteria are satisfied. As shown above, the feedback loop can be performed on the hybridization steps, washing steps, or both.

For example, one or more conditions of the polynucleotide array can initially be of low stringency. If the data corresponding to selected spots does not satisfy the specified criteria, then the conditions of the polynucleotide array can be raised to a medium stringency. If the data corresponding to selected spots still does not satisfy the specified criteria, then the conditions of the polynucleotide array can be raised to high stringency, and so on and so forth until the specified criteria are satisfied.

It should be emphasized that many variations and modifications may be made to the above-described embodiments. For example, the feedback loop described in reference to FIG. 5 can be used to optimize the general conditions applied to the entire array or certain portions thereof. All such modifications and variations are intended to be included herein within the scope of this disclosure and protected by the following claims.

1. A method for performing polymer analysis, comprising:
providing a sample and a polymer array, the polymer array having a plurality of spots;
providing a set of conditions that are selected to generate a response from selected spots;
applying the set of conditions to the sample; and
generating data corresponding to the response from the selected spots.

2. The method of claim 1, further comprising:
modifying the set of conditions applied to the sample until the data satisfy a criteria.

3. The method of claim 1, wherein providing a set of conditions comprises:
providing a set of hybridization conditions.

4. The method of claim 3, wherein providing a set of hybridization conditions comprises:
adjusting the hybridization conditions applied to the sample dynamically until hybridization data satisfies a hybridization criteria corresponding to the selected spots.

5. The method of claim 3, wherein the hybridization conditions are selected from: temperature of the polymer array, pH of the polymer array, a time period of the polymer array, and stringency of the polymer array, and combinations thereof.

6. The method of claim 1, wherein providing a set of conditions comprises:
providing a set of washing conditions.

7. The method of claim 6, wherein providing a set of washing conditions comprises:
adjusting the washing conditions applied to the sample dynamically until a washing data satisfy washing criteria corresponding to the selected spots.

8. The method of claim 6, wherein the washing conditions are selected from: temperature of the nucleic acid array, pH of the nucleic acid array, a time period of the nucleic acid array, and stringency of the nucleic acid array.

9. A method for performing a polymer array analysis, comprising:
providing a sample and a polymer array, the polymer array having a plurality of spots;
providing a set of hybridization conditions and a set of wash conditions that are selected to generate a response for selected spots;
applying the hybridization conditions to the sample;
generating hybridization data corresponding to a first response from the selected spots;
adjusting the hybridization conditions applied to the sample until the hybridization data satisfy hybridization criteria;
applying the wash conditions to the sample;
generating washing data corresponding to a second response from the selected spots;
adjusting the washing conditions applied to the sample until the washing data satisfy washing criteria; and
generating polymer array data.

10. The method of claim 9, wherein the polymer array is selected from a polynucleotide array and a polypeptide array.

11. The method of claim 9, wherein the hybridization conditions are selected from: temperature of the polymer array, pH of the polymer array, a time period of the polymer array, and stringency of the polymer array, and combinations thereof.

12. The method of claim 9, wherein the washing conditions are selected from: temperature of the nucleic acid array, pH of the nucleic acid array, a time period of the nucleic acid array, and stringency of the nucleic acid array.

13. A system for performing polymer analysis, comprising:
a polymer control system operative to:
apply a set of conditions to a sample being analyzed using a polymer array, the polymer array having a plurality of spots, the set of conditions are selected to generate a response from the selected spots;
analyze data corresponding to the selected spots using the polymer control system; and
generate polymer array data.

14. The system of claim 13, wherein the polymer control system is stored on a computer-readable medium.

15. The system of claim 13, wherein the polymer control system comprises:
receptive logic configured to analyze the data.

16. The system of claim 13, wherein the polymer control system is further operative to dynamically adjust the set of conditions applied to the polymer array.

17. The system of claim 13, further comprising:
means for analyzing the data.

18. The system of claim 13, wherein the set of conditions is selected from a set of hybridization conditions and a set of wash conditions.

19. The system of claim 13, further comprising a polymer analysis system comprising the polymer array and the polymer control system, the polymer array selected from a polynucleotide array and a polypeptide array.
